(19) United States
(12) Patent Application Publication
(10) Pub. No.: US 2018/0360541 A1
(43) Pub. Date: Dec. 20, 2018

(54) MEDICAL IMAGE PROCESSING APPARATUS, MEDICAL IMAGE DIAGNOSTIC APPARATUS, AND MEDICAL IMAGE PROCESSING METHOD

(71) Applicant: CANON MEDICAL SYSTEMS CORPORATION, Otawara-shi (JP)

(72) Inventors: Chihiro MORITA, Nasushiobara (JP); Tatsuo MAEDA, Nasushiobara (JP); Shintaro FUNABASAMA, Utsunomiya (JP)

(73) Assignee: CANON MEDICAL SYSTEMS CORPORATION, Otawara-shi (JP)

(21) Appl. No.: 16/007,294

(22) Filed: Jun. 13, 2018

(30) Foreign Application Priority Data
Jun. 15, 2017 (JP) 2017-117785

(51) Int. Cl. A61B 34/10; G06T 7/00; A61B 6/03; A61B 6/00; A61B 17/80; A61B 17/86

(52) U.S. Cl. CPC A61B 34/10; G06T 7/0012; A61B 6/032; A61B 6/505; A61B 6/5217; A61B 17/80; A61B 2090/3762; G06T 2207/30008; G06T 2207/10116; A61B 2034/104; A61B 2034/105; A61B 2034/108; A61B 17/86

(57) ABSTRACT

A medical image processing apparatus according to the present embodiment, configured to support selection of a screw for fixing a plate to a bone, includes processing circuitry. The processing circuitry is configured to: acquire a medical image including a target bone to which the plate is to be fixed; identify the target bone based on the medical image; calculate spatial distribution indicating fragility degree of the target bone, based on pixel values of the target bone; and select a screw to be embedded in the target bone, based on the spatial distribution.

### CROSS-REFERENCE TO RELATED APPLICATION

This application is based upon and claims the benefit of priority from Japanese Patent Application No. 2017-117785, filed on Jun. 15, 2017, the entire contents of each of which are incorporated herein by reference.

### FIELD

Embodiments described herein relate generally to a medical image processing apparatus, a medical image diagnostic apparatus, and a medical image processing method.

### BACKGROUND

There is a method to treat fracture by placing an implant in a body. Implants used for fracture treatment include, for example, a plate placed at a fracture site for supporting or fixing a fractured bone and a screw for screwing the plate to the bone. The screws are sometimes used alone in the case of screwing and fixing the bone.

Various types of plates are provided depending on each fracture site and each bone shape. Further, there have been developed apparatuses and programs for simulating a position of a plate in a fractured bone with the use of a medical image depicting a fracture site. The fracture site is a bone region centered on the deformed or damaged portion of the bone. In the following description, a medical image depicting a fracture site is referred to as a fracture-site image.

Various screws also are provided depending on the type of plate and bone shape. However, the type of screws, diameter and length of screws differ for each fracture site depending on the state, nature, position, or shape of the fractured bone. In the present specification, diameter means diameter of a shaft portion of a screw. In addition, length means the entire length of a shaft portion of a screw. Further, in order to fix one plate to a bone, plural different types of screws are used, and appropriate screws are different depending on the fixed position and direction.

Accordingly, in conventional technology, screws are selected on the basis of knowledge and experience of each doctor. Since screws to be selected are different for each fracture site, selection of screws is one of the time-consuming tasks in a fracture treatment plan. Thus, there has been a demand for a device that supports selection of appropriate screws for a fracture site.

### BRIEF DESCRIPTION OF THE DRAWINGS

In the accompanying drawings:

FIG. 1 is a conceptual configuration diagram illustrating a medical image diagnostic apparatus according to an embodiment;

FIG. 2 is a functional block diagram illustrating a functional configuration of the medical image processing apparatus according to the embodiment;

FIG. 3 is a first flowchart illustrating an operation of the medical image processing apparatus according to the embodiment;

FIG. 4 is a second flowchart illustrating an operation of the medical image processing apparatus according to the embodiment;

FIG. 5 is a schematic diagram illustrating positioning of a plate with respect to a bone;

FIG. 6 is a schematic diagram illustrating distinguishable display of bone fragility on the basis of Misch classification;

FIG. 7 is a schematic diagram illustrating distinguishable display of bone fragility for each screw position to be inserted;

FIG. 8 is a screw table illustrating a list of screws applicable to a plate;

Each of FIGS. 9A and 9B is a schematic diagram illustrating length of a bone along a screw direction to be inserted;

FIG. 10 is a schematic diagram for explaining a display aspect of an applicable screw list;

FIG. 11 is a schematic diagram illustrating a screw direction to be inserted; and

Each of FIGS. 12A to 12C is a schematic diagram illustrating each screw type and a fixed position thereof.

### DETAILED DESCRIPTION

Prior to a description of embodiments of the present invention, first, a description will be given of the problem in screw selection for fixing a plate and the points on which the present inventors focused.

There is a treatment method to fix a fractured bone by placing a plate on the surface of the fractured bone including the fracture site and screwing the plate to the fractured bone with screws. There are various methods for fixing the plate depending on the fracture site and how the bone is fractured, and a doctor selects a method that fits the fracture site and how the bone is fractured out of many plate fixing methods. Further, plates and screws to be used are different depending on each fracture site and how the bone is fractured.

As to selection of a plate, for instance, there are provided devices and programs that can simulate an installed position of a plate by superimposing an image of the plate on a computed tomography (CT) image. By using such devices or programs, a user such as a doctor can select an appropriate plate and determine the plate installed position according to the fracture site.

Screws for fixing a plate vary depending on a fixation method of the plate and an installed position of the plate with respect to a target bone. The fixation method and installed position of the plate differ depending on the fracture site, how the bone is fractured, and the state and character of the bone at the fracture site. In particular, the fixation method of a plate significantly differs depending on character of the target bone such as bone hardness and fragility.

For instance, when the target bone is fragile, screws called locking screws are selected. Each of the locking screws has a thread-cutting part on its head portion and can be fixed to both of the target bone and the plate by screwing the thread-cutting part and the plate to each other. Since the locking screws exert the fixing force not only to the target bone but also to the plate, for instance, the locking screws are used near the fracture site or in the case of a fragile bone.

As described above, character of a bone such as fragility and durability serves as one index for selecting appropriate screws in some cases. In view of the above-described problems and focusing points, the present inventors have worked out a configuration in which fragility degree of the target bone can be determined on the basis of the fracture-site image and appropriate screws can be automatically selected on the basis of the fragility of the target bone.

Hereinafter, a description will be given of a medical image processing apparatus, a medical image diagnostic apparatus, and a medical image processing method, each of which has the above-described innovative configuration, with reference to the accompanying drawings.

A medical image processing apparatus according to the present embodiment, configured to support selection of a screw for fixing a plate to a bone, includes processing circuitry. The processing circuitry is configured to: acquire a medical image including a target bone to which the plate is to be fixed; identify the target bone based on the medical image; calculate spatial distribution indicating fragility degree of the target bone, based on pixel values of the target bone; and select a screw to be embedded in the target bone, based on the spatial distribution.

### 1. Configuration

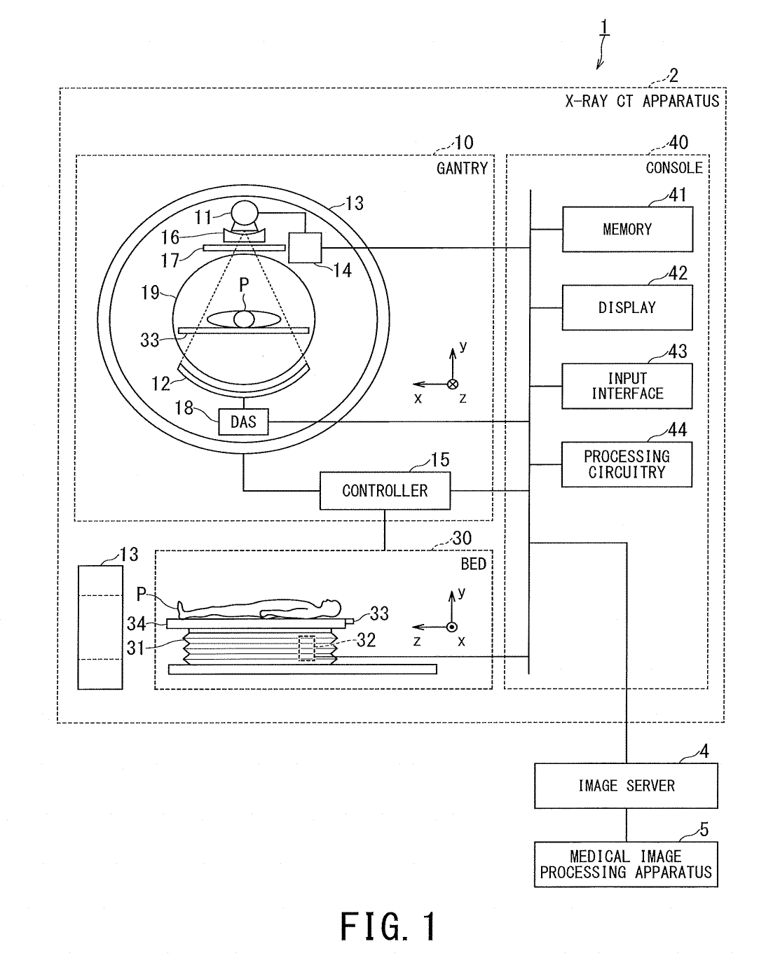

FIG. 1 is a conceptual configuration diagram illustrating a medical image diagnostic apparatus 1 according to one embodiment. The medical image diagnostic apparatus 1 in FIG. 1 includes an X-ray CT apparatus 2, a medical image processing apparatus 5, and an image server 4. The X-ray CT apparatus 2, the medical image processing apparatus 5, and the image server 4 are interconnected via a network such as a local area network (LAN).

Although a description will be given of the medical image diagnostic apparatus 1 equipped with the X-ray CT apparatus 2, the medical image diagnostic apparatus 1 may be equipped with another X-ray apparatus such as a dental CT apparatus and an X-ray angiography apparatus having a C-arm and/or an Q-arm instead of the X-ray CT apparatus 2. The medical image diagnostic apparatus 1 acquires a fracture-site image and a plate image from an X-ray apparatus such as the X-ray CT apparatus 2.

The X-ray CT apparatus 2 includes various types such as a rotate/rotate type (for example, third generation CT) in which an X-ray tube and an X-ray detector integrally rotate around an object, a stationary/rotate type (for example, fourth generation CT) in which a large number of X-ray detection elements arrayed in a ring shape are fixed and only the X-ray tube rotates around the object, and any type can be applied to the present embodiment. In the following, a description will be given of a case where the rotate/rotate type of the third generation is adopted as the X-ray CT apparatus 2 according to the present embodiment.

The X-ray CT apparatus 2 includes a gantry 10, a bed 30, and a console 40. The gantry 10 and the bed 30 are installed in an examination room. The gantry 10 generates X-ray detection data (transmission data) related to an object (for example, a patient) P placed on the bed 30. The console 40 is installed in a control room adjacent to the examination room, and generates and displays a reconstructed image by generating projection data on the basis of the detection data.

The gantry 10 includes an X-ray tube 11, an X-ray detector 12, a rotating frame 13 having an opening 19 in which an imaging region resides, a high-voltage power source 14, a controller 15, a wedge 16, a collimator 17, and a data acquisition system (DAS) 18.

The X-ray tube 11 is a vacuum tube that radiates thermoelectrons from a cathode (filament) to an anode (target) by application of high voltage supplied from the high-voltage power source 14. The configuration of the present embodiment can be applied to both of a single-tube type X-ray CT apparatus and a so-called multi-tube type X-ray CT apparatus in which plural pairs of X-ray tubes and X-ray detectors are mounted on respective rotary rings.

Hardware for generating X-rays is not limited to the X-ray tube 11. For instance, instead of the X-ray tube 11, X-rays may be generated by the fifth generation system that includes a focus coil for converging an electron beam generated from an electron gun, a deflection coil for electromagnetically deflecting the electron beam, and a target ring for generating X-rays by causing the electron beam deflected around the half circumference of the object P to collide.

The X-ray detector 12 detects X-rays that have radiated from the X-ray tube 11 and transmitted through the object P, and outputs an electric signal corresponding to the X-ray amount to the DAS 18. For instance, the X-ray detector 12 includes plural X-ray detection element columns, each of which is composed of plural X-ray detection elements arrayed in the channel direction along one circular arc around the focal point of the X-ray tube 11. For instance, the X-ray detector 12 has a structure in which each of the X-ray detection element columns is composed of plural X-ray detection elements arrayed in the channel direction and those X-ray detection element columns are arrayed in the slice direction (column direction or row direction).

For instance, the X-ray detector 12 is an indirect conversion type detector equipped with a grid, a scintillator array, and a photosensor array. The scintillator array includes plural scintillators, and each scintillator has a scintillator crystal that outputs light with an amount of photons corresponding to incident X-ray amount. The grid is disposed on the X-ray incident side of the scintillator array, and is equipped with an X-ray shielding plate that has a function of absorbing scattered X-rays. The photosensor array has a function of converting the light from the scintillator into an electric signal corresponding to the amount of the light from the scintillator, and has, for example, photosensors such as a photo multiplier tube (PMT).

The X-ray detector 12 may be a direct conversion type detector equipped with semiconductor elements for converting incident X-rays into electric signals.

The rotating frame 13 is an annular frame that supports the X-ray tube 11 and the X-ray detector 12 in the state of facing each other with the object P interposed therebetween and rotates the X-ray tube 11 and the X-ray detector 12 under the control of the controller 15 as described below. In addition to the X-ray tube 11 and the X-ray detector 12, the rotating frame 13 further includes and supports the high-voltage power source 14 and the DAS 18.

The detection data generated by the DAS 18 are transmitted by optical communication from a transmitter having a light emitting diode (LED) provided in the rotating frame 13 to a non-rotating portion of the gantry 10 (for example, a receiver that has photodiodes provided in a non-illustrated fixed frame), and are further transferred to the console 40. The method of transmitting the detection data from the rotating frame 13 to the non-rotating portion of the gantry 10 is not limited to the above-described optical communication, and any method may be adopted for transmitting the detection data as long as it is a non-contact type data transmission. In addition, the above-described non-illustrated fixed frame is a frame that rotatably supports the rotating frame 13.

As described above, the X-ray CT apparatus 2 acquires the detection data for the entire circumference of the object P, that is, detection data over all the angles of 360 degrees for the object P by rotating the rotating frame 13, which supports the X-ray tube 11 and the X-ray detector 12 in the state of facing each other. The reconstruction method of a CT image is not limited to a full-scan reconstruction method using detection data over all the angles of 360 degrees. For instance, the reconstruction method may be a half reconstruction method in which a CT image is reconstructed on the basis of the detection data for half a circumference (180°) plus a fan angle.

The high-voltage power source 14 has an electric circuit such as a transformer and a rectifier. In addition, the high-voltage power source 14 includes a high-voltage generator having a function of generating high voltage applied to the X-ray tube 11, and further includes an X-ray controller for controlling the output voltage according to X-rays radiated by the X-ray tube 13. The high voltage generator may be a transformer type or an inverter type. The high-voltage power source 14 may be provided in the rotating frame 13 described below or may be provided on the fixed frame side of the gantry 10.

The controller 15 has a processor, a memory, and a driving mechanism such as a motor and an actuator. The controller 15 has a function of receiving an input signal from an input interface mounted on the console 40 or the gantry 10 and controlling the operation of the gantry 10 and the bed 30.

For instance, the controller 15 receives an input signal and controls respective components on the basis of the input signal for rotating the rotating frame 13, tilting the gantry 10, and driving the bed 30, for example, moving the table-top 33. The controller 15 achieves the control of tilting the gantry 10 by rotating the rotating frame 13 around the axis in parallel to the x-axis on the basis of inclined angle information (tilt angle information) inputted from the input interface that is mounted on the gantry 10. The controller 15 may be provided in the gantry 10 or in the console 40.

As one possible aspect, the apparatus coordinate system of the X-ray CT apparatus 2 is defined as follows: the y-axis direction is defined as the vertical direction; the z-axis direction is defined as the rotation axis of the rotating frame 13 in the non-tilted state, for example, the longitudinal direction of the table-top 33 of the bed 30; and the x-axis direction is defined as the direction perpendicular to both of the z-axis direction and the y-axis direction.

The wedge 16 is a filter for adjusting amount of X-rays radiated from the X-ray tube 11. Specifically, the wedge 16 is a filter configured to attenuate X-rays radiated from the X-ray tube 11 by causing the X-rays to penetrate the wedge 16 such that the X-rays radiated onto the object P from the X-ray tube 11 have predetermined distribution. For instance, the wedge 16 (wedge filter or bow-tie filter) is a filter obtained by processing aluminum such that the aluminum has a predetermined target angle and/or predetermined thickness. The collimator 17 is a lead plate or the like for narrowing the irradiation range of X-rays transmitted through the wedge 16, and forms a slit by a combination of plural lead plates.

The DAS 18 includes an amplifier configured to perform amplification processing on electric signals outputted from the respective X-ray detection elements of the X-ray detector 12, further includes an A/D converter configured to convert the electric signals into digital signals, and generates detection data. The detection data generated by the DAS 18 are transferred to the console 40.

The bed 30 is an apparatus for placing and moving the object P to be scanned, and includes a base 31, a bed driver 32, a table-top 33, and a support frame 34.

The base 31 is a housing that movably supports the support frame 34 in the vertical direction (y-axis direction). The bed driver 32 is a motor or actuator that moves the table-top 33, on which the object P is placed, in the longitudinal direction (z-axis direction) of the table-top 33. The table-top 33 provided on the upper surface of the support frame 34 is a plate on which the object P is placed.

In addition to moving the table-top 33, the bed driver 32 may move the support frame 34 in the longitudinal direction (z-axis direction) of the table-top 33. Further, the bed driver 32 may move the table-top 33 together with the base 31 of the bed 30. In the case of applying the present invention to CT in a standing position, a method of moving the movement mechanism of the object P corresponding to the table-top 33 may be adopted. When imaging is performed with relative change of positional relationship between the table-top 33 and the imaging system of the gantry 10, such as a helical scan imaging and scanography for positioning, the relative change of the positional relationship may be performed by driving the table-top 33, performed by running the fixed frame of the gantry 10, or performed by combination of those.

The console 40 includes a memory 41, a display 42, an input interface 43, and processing circuitry 44. Although it is assumed that the console 40 executes all the functions with a single console in the following description, these functions may be executed by plural consoles.

The memory 41 has a configuration including a processor-readable recording medium such as a hard disk, an optical disk, and a semiconductor memory element including a random access memory (RAM) and a flash memory.

Projection data and reconstructed image data generated by the X-ray CT apparatus 2 may be stored in the memory 41. In addition, the projection data and the reconstructed image data generated by the X-ray CT apparatus 2 may be stored in the image server 4 that is connectable to the X-ray CT apparatus 2 via the network. Similarly, some or all of the programs and data in the recording medium of the memory 41 may be downloaded by communication via a network or may be given to the memory 41 via a portable storage medium such as an optical disc.

The display 42 displays various types of information. For instance, the display 42 outputs a medical image (CT image) generated by the processing circuitry 44 and a graphical user interface (GUI) for receiving various operations from the user. The display 42 is, for example, a liquid crystal display, a cathode ray tube (CRT) display, or an organic light emitting diode (OLED) display.

The input interface 43 receives various input operations from a user, converts the received input operations into electric signals, and outputs the electric signals to the processing circuitry 44. For instance, the input interface 43 receives, from a user, setting information such as imaging conditions for acquiring projection data, reconstruction conditions for reconstructing a CT image, and image processing conditions for processing a CT image. For instance, the input interface 43 is realized by a mouse, a keyboard, a trackball, a switch, a button, and a joystick.

The processing circuitry 44 controls the entire operation of the X-ray CT apparatus 2. In addition, the processing circuitry 44 executes preprocessing such as correction processing on the detection data outputted from the gantry 10, and reconstructs the detection data subjected to the preprocessing so as to generate CT image data. Further, the processing circuitry 44 executes image processing for generating tomographic image data and three-dimensional image data of an arbitrary cross-section by applying a known method to the CT image data.

The processing circuitry 44 may be configured of dedicated hardware or may be configured to implement various functions described below by software processing with the use of a built-in processor. As one possible aspect, a description will be given of a case where the processing circuitry 44 implements various functions by software processing with the use of a processor.

The above-described term “processor” means, for example, a circuit such as a special-purpose or general-purpose central processing unit (CPU), a special-purpose or general-purpose graphics processing unit (GPU), an application specific integrated circuit (ASIC), a programmable logic device, and a field programmable gate array (FPGA). The above-described programmable logic device includes, for example, a simple programmable logic device (SPLD) and a complex programmable logic device (CPLD). The processing circuitry 44 implements respective functions by reading out programs stored in the memory 41 and executing the programs. Additionally or alternatively, the processing circuitry 44 implements the respective functions by reading out programs stored in its own processor and executing the programs.

Further, the processing circuitry 44 may be configured of a single processor or may be configured of a combination of plural processors that are independent of each other. In the latter case, plural memories may be provided for the respective processors so that programs executed by each processor are stored in the memory corresponding to this processor. As a further modification, one memory may collectively store all the programs corresponding to the respective functions of the plural processors.

In addition, the functions implemented by the processing circuitry 44 may be implemented by an integrated server other than the console 40. The integrated server is a computer that collectively processes detection data acquired in plural modalities. In the embodiment, the server is a computer that has a memory and a processor and provides its own functions and data with respect to a client computer via a network. Further, the server may be composed of plural physical servers as a virtual server or may be a cloud server that is provided outside the facility and is connectable to the console 40 via a wide-area network.

The medical image processing apparatus 5 may be configured to be able to execute image processing that can be executed by the console 40, such as image reconstruction. Similarly, the console 40 may be configured to be able to execute processing of generating a treatment plan for a fracture site, which can be executed by the medical image processing device 5. The image processing and processing of generating a treatment plan may be simultaneously executed by both of the console 40 and the medical image processing apparatus 5.

The image server 4 stores medical images acquired by the X-ray CT apparatus 2 and other modalities. The image server 4 may store three-dimensional image data such as STL data of an implant in addition to medical images.

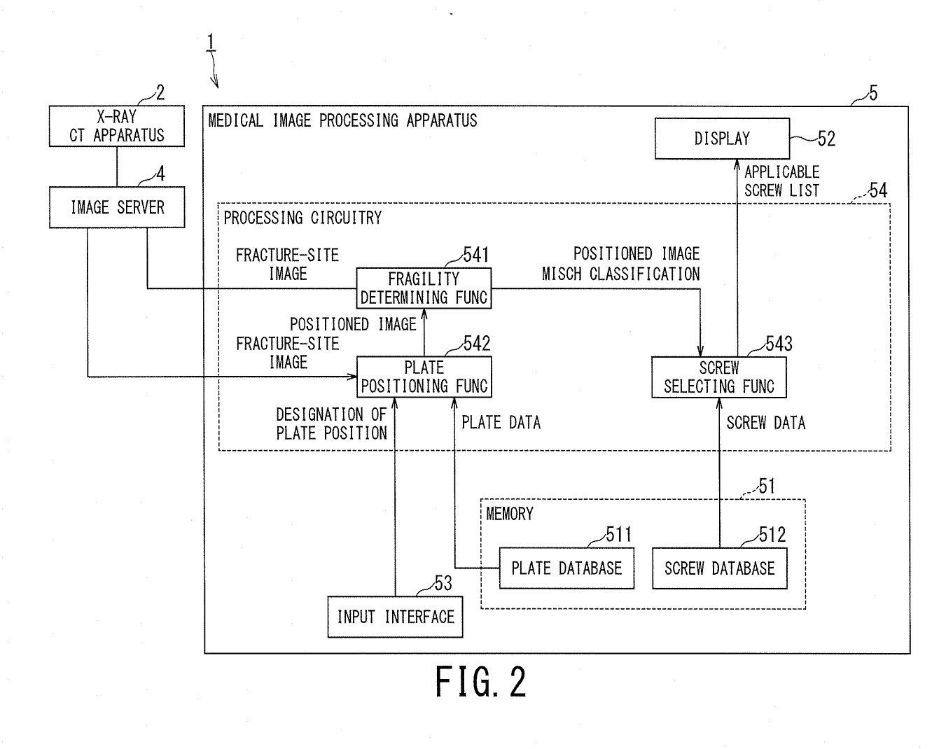

The medical image processing apparatus 5 is composed of, for example, a personal computer, and is an apparatus for a user such as a doctor to prepare a treatment plan for a fracture site. The entire system may be configured such that display and input of a treatment plan are executed from a thin-client and the processing of preparing a treatment plan is executed by the medical image processing apparatus 5 functioning as the server of the thin-client. The configuration of the medical image processing apparatus 5 will be described in detail with reference to FIG. 2.

FIG. 2 is a functional block diagram illustrating a functional configuration of the medical image processing apparatus 5 according to the present embodiment. The medical image processing apparatus 5 includes a memory 51, a display 52, an input interface 53, and processing circuitry 54.

The memory 51 has the same configuration as the memory 41 of the X-ray CT apparatus 2, and stores a plate database 511 and a screw database 512.

The plate database 511 is a database in which information on plates, that is, information on implants used for treating fracture is accumulated. The plate database 511 has plural data units, in each of which a plate ID (identification) number and plural information items indicating features of the plate corresponding to this plate ID number are associated with each other. For instance, the features of each plate include information items such as the name of the part of the target bone on which the plate is placed, the size of the plate, the number of holes provided on the plate for inserting screws.

Further, the plate database 511 may store plate image data. The plate image data includes three-dimensional image data such as STL data and X-ray image data obtained by imaging the plate.

Note that the plate image data may be stored in the image server 4 or may be stored in another external storage device. The medical image processing apparatus 5 may be configured such that plate image data are downloaded from the image server 4 or the external storage device in accordance with selection of the plate ID by a user.

The screw database 512 is a database in which information on screws, the information being on implants used for treating fracture is accumulated. For instance, the screw database 512 is composed of table data in which each of the plates is associated with plural information items indicating features of screws. The table data include plural data units, in each of which a screw ID is associated with plural information items such as a screw type, diameter, length, and information on the state of applicable bones. Details of the table data will be described below with reference to FIGS. 9A and 9B.

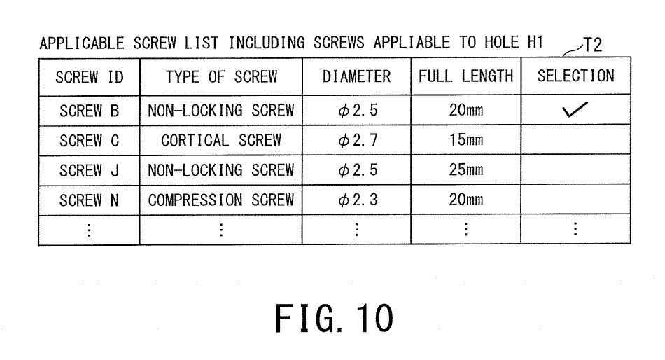

The display 52 has the same configuration as the display 42 of the X-ray CT apparatus 2, and displays a fracture-site image of the object P and a three-dimensional image of the plate under the control of the processing circuitry 54. In addition, a biological index indicative of character of the bone at the fracture site is superimposed and displayed on the fracture-site image. Further, the display 52 displays a screw list that can be embedded in the fracture site. As to display aspects of the screw list that can be embedded in the fracture site to be displayed on the display 52, it will be described below in detail with reference to FIG. 10.

The input interface 53 has the same configuration as the display 42 of the X-ray CT apparatus 2, receives an input from a user such as a doctor, and outputs it to the processing circuitry 54. The input interface 53 receives inputs such as the plate ID of the selected plate and the position designation information of the plate. Further, the input interface 53 receives selection information on the screws selected by a user on the basis of the screw list, which can be embedded in the fracture site, displayed on the display 52.

The processing circuitry 54 has an acquisition function of acquiring a fracture-site image from the image server 4 via a network, and performs image processing on the fracture-site image so as to select an appropriate plate and screws. The processing circuitry 54 has the same configuration as the processing circuitry 44 of the X-ray CT apparatus 2. In the following, a description will be given of a case where the processing circuitry 54 achieves various functions by causing its processor to execute software processing.

The processing circuitry 54 implements a fragility determining function 541, a plate positioning function 542, and a screw selecting function 543 by executing a medical image processing program stored in the memory 51.

The fragility determining function 541 determines a biological index indicating the character of the bone for each pixel of the fracture-site image, on the basis of the fracture-site image acquired from the image server 4 and/or an external storage device. Here, the character of the bone is bone quality such as fragility and hardness of the bone. Biological indexes indicating the character of the bone include, for example, Misch classifications based on CT values.

In the Misch classification, the character of the bone is classified into five stages on the basis of a CT value of each pixel of a medical image. Specifically, in the Misch classification, each pixel is classified into one of five groups in order of hardness. Each pixel is classified into “D5” when its CT value is larger than 1250 HU (Hounsfield Unit), classified into “D4” when its CT value is larger than 850 and smaller than 1250 HU, classified into “D3” when its CT value is larger than 350 HU and smaller than 850 HU, classified into “D2” when its CT value is larger than 150 HU and smaller than 350, and classified into “D1” when its CT value is smaller than 150 HU. Note that “D1” indicates a portion that is not a bone.

The CT value is associated with the X-ray attenuation coefficient of the tissue, and is a relative value under the premise that 0 HU indicates water and −1000 HU indicates air. Since the CT value is obtained from the X-ray attenuation coefficient, the CT value can be calculated from the pixel value of the medical image acquired by using X-rays. For instance, the CT value can be calculated also in a medical image acquired by an X-ray angiography apparatus or the like.

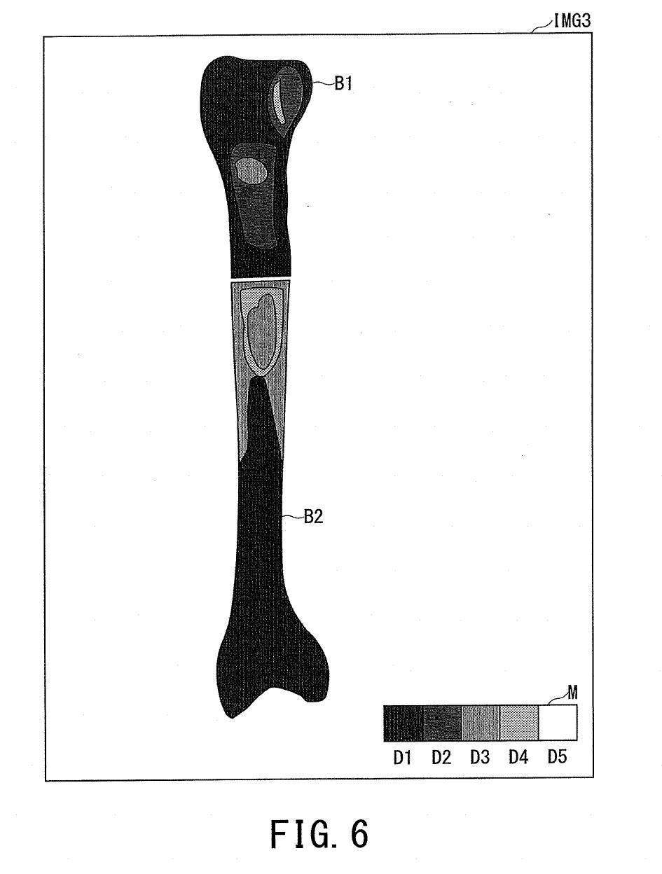

The fragility determining function 541 calculates spatial distribution indicating fragility degree of the bone on the basis of the Misch classification. For instance, the fragility determining function 541 calculates the spatial distribution by determining the fragility of the bone including the fracture site in the fracture-site image for each pixel of the bone. The fragility determining function 541 may distinguishably display bone fragility determined for each pixel on the basis of the Misch classification by using one or plural chromatic colors. For instance, the fragility determining function 541 may display distribution of bone fragility on a medical image on the basis of a color map in which a color corresponding to the Misch classification determined for each pixel of the fracture-site image is assigned to every pixel of the fracture-site image. Details of the Misch classification performed by the fragility determination function 541 will be described below with reference to FIG. 6.

The biological index indicating bone fragility is not limited to the Misch classification. For instance, bone fragility may be determined on the basis of tendency of the CT values of the entirety of the inside of the bone. Specifically, when the average value or the variance value of the CT values on the straight line connecting the center of the bone and the surface of the bone is lower than the predetermined threshold value, it is determined that the bone is fragile. Further, the fragility determining function 541 may determine bone fragility in the fracture site by reflecting distance from the fracture line and the number of fracture lines.

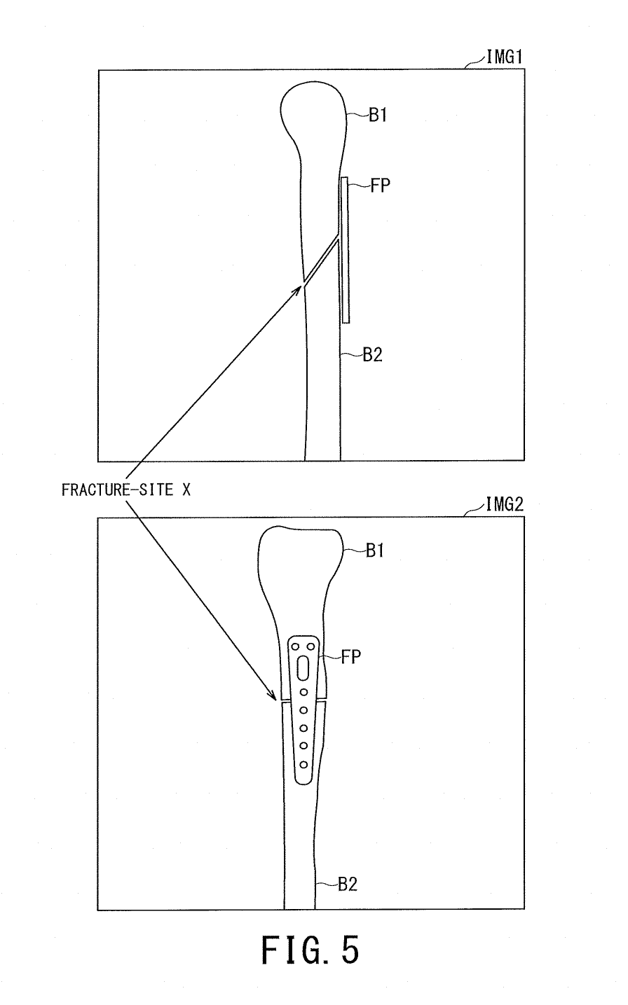

The plate positioning function 542 determines the installed position of the plate by simulating the position of the bone having the fracture site and the position of the plate placed on the bone. The plate positioning function 542 acquires the fracture-site image from the image server 4, superimposes the plate image on the fracture-site image, and displays it on the display 52. The plate image may be stored in the plate database 511 or may be downloaded from an external storage device such as the image server 4 on the basis of the plate ID selected by a user so as to be displayed. Details of positioning between the plate and the target bone will be described below with reference to FIG. 5.

The screw selecting function 543 selects at least one screw type or screw ID that conforms to the installed position of the plate on the basis of bone fragility at this installed position. The screw selecting function 543 refers to the screw database 512, identifies the screw ID that is suitable to the plate to be installed and the bone fragility, and generates an adaptable screw list.

Further, the screw selecting function 543 compares the length of the target bone along a direction to be inserted into the target bone with the length of the screw so as to determine whether the screw penetrates the bone or not. Here, “penetrate” means that the tip of the screw protrudes from the bone surface (periosteum) that is the opposite side to a screw position to be inserted. As to details of the method of generating an applicable screw list and the method of determining whether the screw penetrates the bone or not, they will be described below with reference to FIGS. 9A, 9B, and 10.

### 2. Operation

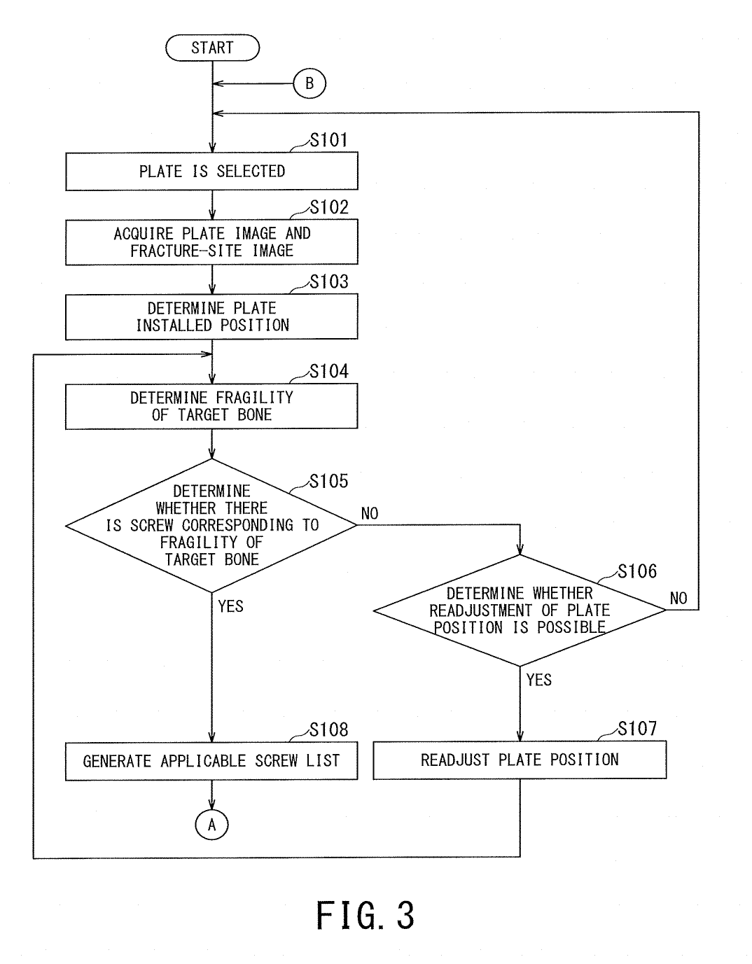

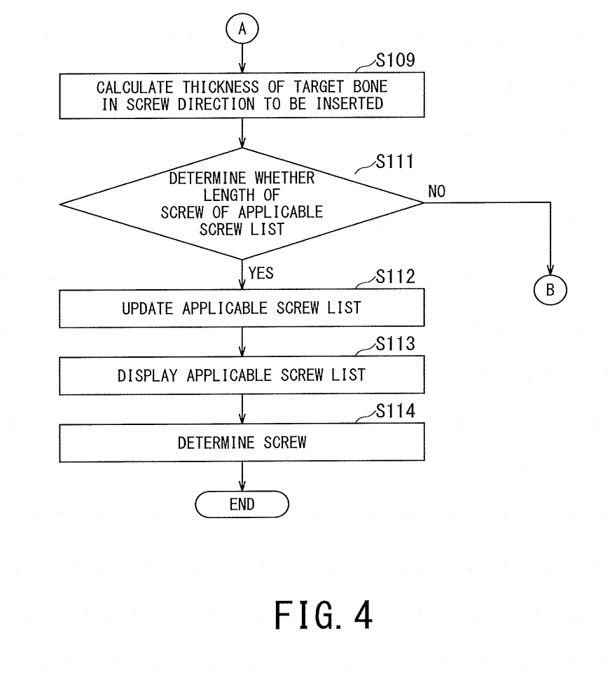

FIG. 3 is a first flowchart illustrating an operation of the medical image processing apparatus 5 according to the present embodiment. FIG. 4 is a second flowchart illustrating an operation of the medical image processing apparatus 5 according to the present embodiment. Hereinafter, an operation performed by the medical image processing apparatus 5 will be described on the basis of the step number in the flowcharts of FIGS. 3 and 4 by referring to FIGS. 5 to 8 as required.

First, on the basis of the flowchart of FIG. 3, a description will be given of an operation of determining fragility of the target bone, on which the plate is placed, and generating an applicable screw list.

In the step S101, a user selects a plate that fits the fracture site position of the object P from plural plate types. For instance, selection of the plate may be performed in such a manner that a user designates a desired plate from the plate type list displayed on the display 52 or the plate positioning function 542 searches the plate database 511 on the basis of information on the object P such as examination information and medical record information and causes the display to display the plate suitable to the fracture-site position.

In the next step S102, the plate positioning function 542 acquires the plate image depicting the designated plate from the plate database 511. The plate positioning function 542 may acquire the plate image from the image server 4. Further, the plate positioning function 542 may acquire the fracture-site image from the image server 4. The plate positioning function 542 displays the acquired fracture-site image and the acquired plate image on the display 52.

Note that acquisition and display of the fracture-site image may be executed before the step S102. For instance, the fracture-site image acquired before the step S101 by the acquisition function may be displayed on the display 52 so that a user refers to the fracture-site image and selects an appropriate plate for the fracture site.

In the next step S103, the plate positioning function 542 performs positioning of the plate with respect to the fracture site of the fracture-site image, and determines the plate installed position.

FIG. 5 is a schematic diagram illustrating the positioning of the plate with respect to the target bone. The upper part of FIG. 5 is a sagittal cross-section IMG1 of the fracture-site image of the object P. The lower part of FIG. 5 is a coronal cross-section IMG2 of the fracture-site image of the object P. In FIG. 5, the fractured bone is separated into the bone B1 and the bone B2 at the fracture site X. The bone crack or fissure observed in the fracture site X is a fracture line.

FIG. 5 illustrates a case where the plate FP is positioned on the right side of the fracture site X in the sagittal cross-section IMG1 and is positioned on the front side of the bone (the plane parallel to the coronal cross-section) in the coronal cross-section IMG2. As shown in the lower part of FIG. 5, the plate FP is provided with plural holes. These holes are for inserting screws, the screws being inserted into respective positions of the holes such that the plate is fixed to the bone.

The plate positioning function 542 causes the display 52 to display plural arbitrary cross-sections of the fracture-site image, and performs positioning between the fractured bone and the plate. The positioning of the plate may be performed in such a manner that a user moves the plate image displayed on the display 52 by using an input device such as a mouse. In addition, the plate positioning function 542 may automatically identify the fractured bone by image processing and perform positioning such that the surface shape of the fractured bone matches the surface shape of the plate. Since the positioning between the plate and the bone having the fracture site may be the same as conventional technology, its detailed description is omitted.

The foregoing is the description of the positioning between the plate and the target bone. Returning to FIG. 3, the description of the flowchart will be continued.

In the next step S104, the fragility determining function 541 acquires the positioned image, in which positioning between the plate and the target bone is completed, from the plate positioning function 542, and determines fragility of the target bone, on which the plate is placed, for each pixel of the target bone. Note that the fragility determining function 541 may acquire the fracture-site image from the image server 4 and determine the fragility of all the bones depicted in the fracture-site image. In addition, the fragility determination processing in the step S104 may be executed for all the bones depicted in the fracture-site image in parallel to the positioning between the plate and the target bone or before the positioning.

The fragility determining function 541 determines bone fragility by the Misch classification based on a CT value of each pixel. The fragility determining function 541 may generate a color map in which a color corresponding to the Misch classification determined for each pixel of the fracture-site image is assigned to each pixel of the target bone to be subjected to positioning with the plate, so as to cause the display 52 to display the color map. A user may prepare a treatment plan for fracture by using support information such as the color map and determine the fixing method of the plate.

FIG. 6 is a schematic diagram in which bone fragility is distinguishably displayed on the basis of the Misch classification. FIG. 6 is one case of a color map in which colors corresponding to the Misch classification are assigned to the respective pixels of the target bone to be subjected to positioning with the plate. The lower right of FIG. 6 is a legend indicating identification display composed of D1 to D5 under the Misch classification.

In the Misch classification shown in FIG. 6, gray scale is used in such a manner that a thicker black color is assigned to a pixel of a harder bone part. In other words, D1 indicative of the hardest bone part among D1 to D5 is shown by the darkest black in gray scale, D2 indicative of the second hardest bone part is shown by the second darkest black in gray scale, and D3 to D5 are shown by respective lighter colors in gray scale as the bone becomes fragile. However, the identification display of the Misch classification is not limited to the above-described gray scale, and chromatic colors such as red, blue, and green may be assigned according to the Misch classification for distinguishably displaying respective pixels in terms of bone fragility.

The fragility determining function 541 determines the Misch classification on the basis of the CT value of each pixel. That is, the fragility determining function 541 determines the Misch classification for each voxel of CT image data that are volume data. The fragility determining function 541 may determine the Misch classification on the basis of pixel values of an arbitrary tomographic image. In addition, the fragility determining function 541 may interpolate the Misch classification determined for each voxel according to the arbitrary tomographic image so as to obtain the Misch classification for each pixel on this tomographic image.

FIG. 6 illustrates a color map in which colors corresponding to the Misch classification are assigned to respective pixels of the tomographic image of the coronal cross-section of the target bone(s) to be subjected to positioning with the plate. In this color map, the Misch classification near the fracture site X of the bone B2 is D2 to D4, and the color map indicates that the partial bone-region near the fracture site X of the bone B2 is fragile.

For instance, the color map display of bone fragility shown in FIG. 6 is also effective as support information for assisting a user in selecting screws in the case of fixing a target bone by using only screws without using a plate. Supportive indications such as a color map showing distribution of fragility and fragility of the entire bone are also useful in a treatment plan for fracture, in the case of determining a method of fixing a plate.

When the Misch classification is determined for each voxel, the fragility determining function 541 may statistically process the Misch classification so as to determine the fragility of the entirety of the fractured bone. In addition, the fragility determining function 541 may statistically process the Misch classification so as to determine bone fragility in a predetermined region.

The above-described statistical processing means processing of comprehensively determine bone fragility within the predetermined region by analyzing tendency of data with the use of statistical values such as the additional value, the average value, the median value, the variance, and the standard deviation of the Misch classification and CT values of respective pixels.

The above-described predetermined region may be, for example, the entire installation surface of the plate and the bone, a region of the hole(s) of the plate as a screw position to be inserted, or a peripheral region around the hole of the plate.

The region around the hole of the plate may be a region of the surface of the bone directly beneath the holes of the plate or all of the bone region directly beneath the hole of the plate. In other words, the fragility determining function 541 may statistically process the Misch classification determined for each voxel in the cylindrical region of the bone immediately under the hole in the plate so as to determine the Misch classification in the cylindrical region.

In addition, the region around the hole of the plate is a region of the surface of the bone including the region outside the contour of the hole in the plate. The region around the hole of the plate also includes a three-dimensional region of the bone, which is obtained by enlarging the cylindrical region of the bone immediately under the hole of the plate to the area outside the outline of the hole of the plate. In this case, the shape of the solid may be immediately, spherical, or cubic. In this manner, the fragility judgment function 541 determines the spatial distribution of bone fragility.

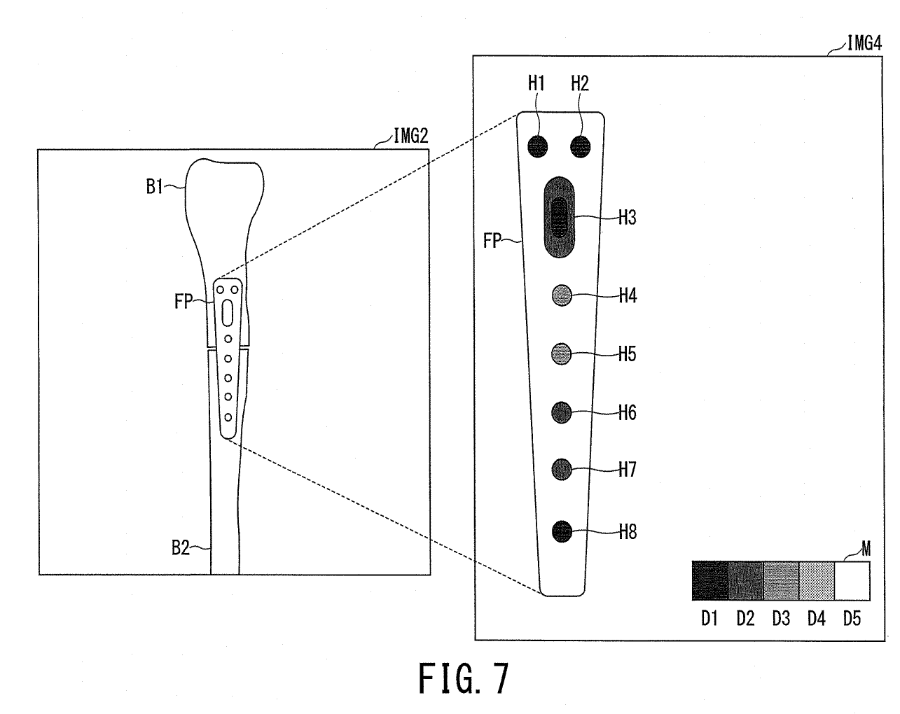

FIG. 7 is a schematic diagram illustrating distinguishable display of bone fragility for each screw position to be inserted. FIG. 7 shows a color map in which a color corresponding to the Misch classification determined for each pixel is assigned to each hole as a screw position, provided on the plate FP. In the lower right part of FIG. 7, a legend of the Misch classification is shown.

The plate FP has eight screw positions to be inserted, of holes H1 to H8. Dark gray-scale hatching corresponding to D1 is assigned to the holes H1, H2, and H8. This indicates that the respective partial bone regions directly beneath the holes H1, H2, and H8 are hard. Light gray-scale hatching corresponding to D3 is assigned to the holes H4 and H5. This indicates that the respective partial bone regions directly beneath the holes H4 and H5 are fragile.

Both the holes H4 and H5 are the holes closest to the fracture site X. For instance, the fragility determining function 541 may perform weighting on the Misch classification depending on whether it is a hole close to fracture site X or not. By the weighting, holes close to the fracture site X are determined to be more fragile than the Misch classification based on CT values, while holes far from the fracture site X are determined to be harder than the Misch classification based on CT values by the weighting. Whether it is close to the fracture site X or not is determined on the basis of distance from the fracture line in the fracture site X. Further, the weighting coefficient may be not only the distance from the fracture line but also length and number of fracture lines. The weighting coefficient may be previously stored in the memory 51. The above-described “previously” means before the execution of the fragility determination processing.

The fragility determining function 541 may divide at least one hole into plural regions according to size of this hole so as to determine fragility in each of the divided region. For instance, the hole H3 is larger in area than the other holes. In such a case, several screws may be inserted into the respective regions of this hole H3. Thus, a hole may be divided into plural regions and fragility may be determined for each of the divided regions. Specifically, fragility may be determined by dividing the entire area of the hole into the periphery of the hole and the center of the hole. In the case of FIG. 7, it is determined that the center of the hole H3 is determined as D5 and the periphery of the hole H3 is determined as is D4.

Note that the fragility determining function 541 may determine the fragility in the entire area of each hole for each pixel and display distribution of fragility in the entire area of each hole as a color map. Additionally, plural pixels may be collectively statistically processed, and distribution of fragility in the entire area of each hole may be displayed as a color map.

The foregoing is the description of the fragility determination processing. Returning to FIG. 3, the description of the flowchart will be continued.

In the next step S105, the screw selecting function 543 determines whether there is a screw corresponding to the fragility of the target bone determined by the fragility determining function 541. The screw selecting function 543 refers to the screw database 512 so as to identify the plate to be used and the screw that is suitable to the fragility of the target bone determined as described above. When the screw selecting function 543 determines that there is a screw applicable to the target bone, the processing proceeds to the step S108. Conversely, when the screw selecting function 543 determines that there is not any applicable screw, the processing proceeds to the step S106.

In the step S106, the plate positioning function 542 determines whether readjustment of the plate position is possible or not. Whether the readjustment of the plate position is possible or not may be determined by the plate positioning function 542 depending on the length of the plate with respect to the target bone and the installed position of the plate. Further, possibility of the readjustment of the plate position may be determined on the basis of number of adjustments of the plate position, as described below.

Specifically, when the number of adjustments of the plate position exceeds a predetermined threshold value, the plate positioning function 542 determines that the position of the plate cannot be readjusted. When the adjustment of the plate position is executed plural times, it is more effective to select another plate than finely readjusting the plate position, in some cases. Thus, when the plate positioning function 542 determines that the position of the plate cannot be readjusted, the processing returns from the step S106 to the step S101 in which a user reselects another plate.

Conversely, when the plate positioning function 542 determines that the position of the plate can be readjusted, the processing proceeds from the step S106 to the step S107.

In the next step S107, the plate positioning function 542 cause the display 52 to display the plate image and the fracture-site image, and changes the position of the plate. After the processing of this step S107, the processing returns to the step S104. In the step S104, bone fragility at the plate position after the fine readjustment is determined. In the next step S105, the screw selecting function 543 identifies the screw that is suitable to the target bone, on the basis of the updated bone fragility determined in the above-described manner. When it is determined in the step S105 that there is an applicable screw, the processing proceeds to the step S108.

In the step S108, the screw selecting function 543 generates an applicable screw list and causes the display 52 to displays the list

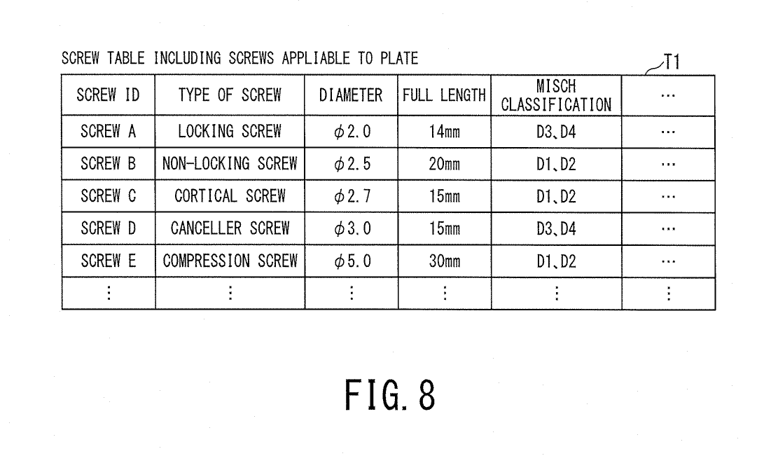

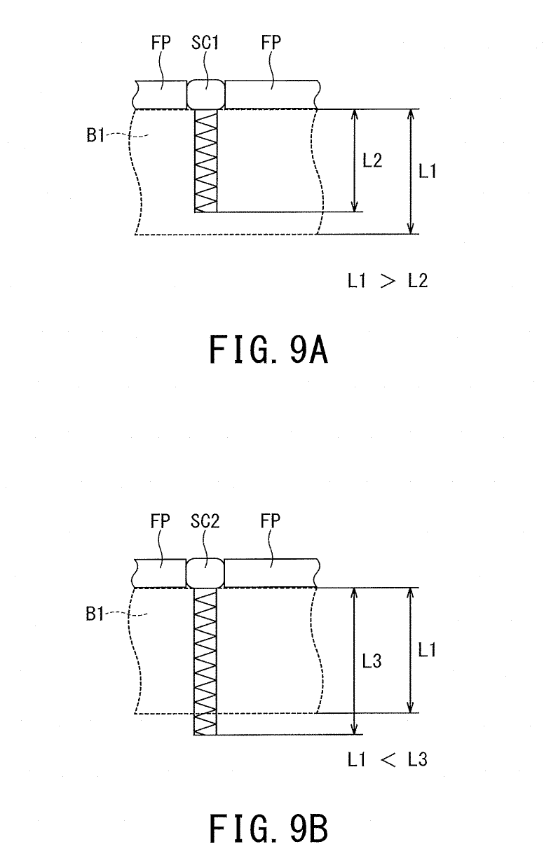

The foregoing is the description of the flowchart of FIG. 3. Hereinafter, the method of specifying applicable screws by the screw selecting function 543 will be described with reference to FIG. 8.

FIG. 8 is a table illustrating a screw table that can be applied to the plate. In the screw database 512, table data of screws are stored for each type of plate. Each row of the table data T1 shows a screw ID, a screw type, diameter, length, and the Misch classification from the left.

The second row of the screw table T1 indicates data of the screw A that is a locking screw in terms of screw type, and has a diameter of φ 2.0 and a length of 14 mm, and is classified into “D3, D4” under the Misch classification. Similarly, the third row of the screw table T1 indicates data of the screw B that is a non-locking screw in terms of screw type, and has a diameter of φ 2.5 and a length of 20 mm, and is classified into “D1, D2” under the Misch classification. The fourth row of the screw table T1 indicates data of the screw C that is a cortical screw, and has a diameter of φ 2.7 and a length of 15 mm, and is classified into “D1, D2” under the Misch classification. The fifth row of the screw table T1 indicates data of the screw D that is a canceller screw, and has a diameter of φ 3.0 and a length of 15 mm, and is classified into “D3, D4” under the Misch classification. The sixth row of the screw table T1 indicates data of the screw E that is a compression screw, and has a diameter of φ 5.0 and a length of 30 mm, and is classified into “D1, D2” under the Misch classification.

The locking screw is a screw that has a thread-cutting part on the head portion and can fix itself to both the target bone and the plate by screwing the thread-cutting part and the plate to each other. A non-locking screw is a screw that has the thread-cutting part only in the shaft portion does not have the thread-cutting part on the head portion.

The cortical screw is a screw that is narrow in pitch at its shaft portion and exerts its fixing force at a cortical bone portion where the bone is hard. The canceller screw is a screw that is wide in pitch at its shaft portion and exerts its fixing force at a sponge bone portion where the bone is soft. The compression screw is a screw used for fixing fractured bones to each other and/or for attracting bones to each other.

The screw table T1 is not limited to the aspect of FIG. 8, and may include information items other than the features of the screws described in FIG. 8. For instance, the screw table T1 may include shape features of each screw such as (a) whether its screw direction to be inserted is a multi-shaft or a single shaft, (b) whether it is a complete screw type in which the entire shaft portion has a thread-cutting part or not, and (c) whether it is an incomplete screw type having a thread-cutting part only at the screw tip portion of the shaft or not. Further, the screw table T1 may include physical features of each screw such as fixing force of the screw against the target bone and whether the fixing force of the plate with respect to the target bone to be exerted by the screw is strong or weak.

For instance, the screw selecting function 543 may identify a screw that is suitable to the target bone for each hole of the plate, on the basis of the bone fragility for each hole indicating the screw position as shown in FIG. 7. For instance, the Misch classification of the hole H1 is D1. Further, it is assumed that the diameter of the hole is φ 3.0. In this case, the screw selecting function 543 proposes the screw B and the screw C to a user as applicable screws on the basis of the screw table T1.

The Misch classification of the hole H4 is D3. When the diameter of the hole is assumed to be φ 3.0 similarly to the hole H1, the screw selecting function 543 proposes the screw A to a user as an applicable screw on the basis of the screw table T1.

In this manner, the screw selecting function 543 selects at least one applicable screw, which is suitable to the target bone for each hole of the plate, from the screw table T1 so as to propose the selected screw to a user. Further, plural screw IDs may be proposed for one hole, and the respective components of the medical image processing apparatus 5 may be configured such that a user can select an appropriate screw from the plurality of proposed screws.

Next, a description will be given of the operation of determining whether the screw is too long to penetrate the bone or not, on the basis of the flowchart of FIG. 4.

In the step S109, the screw selecting function 543 calculates the thickness (length) of the target bone along the screw direction to be inserted. In the case of a single-shaft screw, the screw direction is perpendicular to the surface of the plate.

In the step S109, the screw selecting function 543 determines whether the length of each screw included in the applicable screw list selected on the basis of fragility is shorter than the length of the target bone along the screw direction to be inserted or not. The screw selecting function 543 determines the length of the screw selected by the user.

Note that the processing of the step S109 may be executed before the processing of the step S108. That is, the screw selecting function 543 may generate an applicable screw list that satisfy both conditions in terms of bone fragility described in the step S105 and bone thickness determined in this step S109.

When it is determined in the step S111 that respective lengths of all the screws in the applicable screw list selected on the basis of fragility are equal to or longer than the length of the target bone, the processing returns to the step S101 in which another plate is selected. Conversely, when it is determined in the step S111 that respective lengths of all the screws in the applicable screw list selected on the basis of fragility are shorter than the length of the target bone, the processing proceeds to the step S112.

In the next step S112, the screw selecting function 543 updates the applicable screw list selected on the basis of fragility, and generates the final version of the applicable screw list consisting of screws, each of which is not too long to penetrate the target bone but appropriate in terms of length.

In the next step S113, the screw selecting function 543 causes the display 52 to display the final version of the applicable screw list.

In the next step S114, a user determines the screw to be used for treatment.

The foregoing is the description of the flowchart of FIG. 4. Hereinafter, referring to FIGS. 9A 9B and 10, a description will be given of a method of generating the applicable screw list on the basis of bone length and its display method.

Each of FIGS. 9A and 9B is a schematic diagram illustrating the thickness of the target bone in a screw inserted direction. In each of FIGS. 9A and 9B, the region indicated by the broken line shows the bone B1, and FIGS. 9A and 9B are cross-sectional views of the target bone in the screw inserted direction. FIG. 9A shows a case where the screw SC1 is inserted in the target bone, and FIG. 9B shows a case where the screw SC2 having a longer shaft length than the screw SC1 is inserted at the same screw inserted position as the screw SC1 shown in FIG. 9A.

The length L2 of the shaft portion of the screw SC1 is shorter than the length L1 of the target bone B1 in the screw inserted direction. Contrastively, the length L3 of the shaft portion of the screw SC2 is longer than the length L1 of the target bone B1 in the screw inserted direction, and the screw tip of the screw SC2 penetrates the target bone B1.

The screw selecting function 543 selects a screw that does not penetrate the target bone, from the applicable screw list, and updates the list.

FIG. 10 is a schematic diagram illustrating a display aspect of the applicable screw list. FIG. 10 shows the applicable screw list T2 for the hole H1. The partial bone region immediately under the hole H1 is hard and classified into D1 under the Misch classification, and the diameter of the hole H1 is φ 3.0. The length L1 of the target bone along the screw inserted direction is 30 mm. In this case, the screw used for the partial bone region classified into D1 of the Misch classification is selected from the table such that the following conditions are satisfied. That is, its screw type should be any of a non-locking screw, a cortical screw, and a compression screw, its diameter should be smaller than φ 3.0, and the length of its shaft portion should be shorter than 30 mm.

For instance, the screw selecting function 543 generates the applicable screw list T2 including a screw ID, a screw type, a diameter and a length for each applicable screw. Each row of the applicable screw list T2 may be provided with an additional column for accepting user's selection. FIG. 10 shows a case where a check mark is displayed in the selection column in the rightmost column of the second row of the applicable screw list T2, and a user selects the screw B.

Such a applicable screw list T2 may be generated for each screw position to be inserted, of the plate and displayed on the display 52. Further, when a user selects the screw position, the applicable screw list for the selected screw position may be displayed to allow the user to determine the screw to be used for each screw position.

According to the medical image processing apparatus 5 of the present embodiment as described above, a user can easily determine the screws to be used for treatment by merely selecting the desired screws from the proposed screws. In addition, since selection of screws can be semi-automated, time required for selecting screws can be shortened. Further, by using bone fragility as an index value, it is possible to rationally determine the type of screw, which is conventionally determined on the basis of the knowledge and experience of a doctor, depending on the index value.

Although a description has been given of the case where the screw direction to be inserted is perpendicular to the surface of the plate, the screw direction is not limited to the direction perpendicular to the surface of the plate. For instance, in the case of using a multi-shaft screw and a plate that can be used in combination with the multi-axis screw, a user can insert the multi-shaft screw at an arbitrary angle.

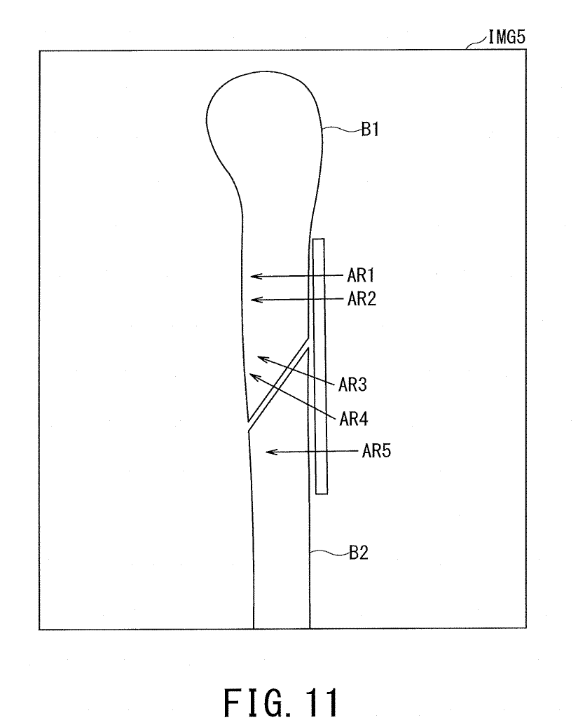

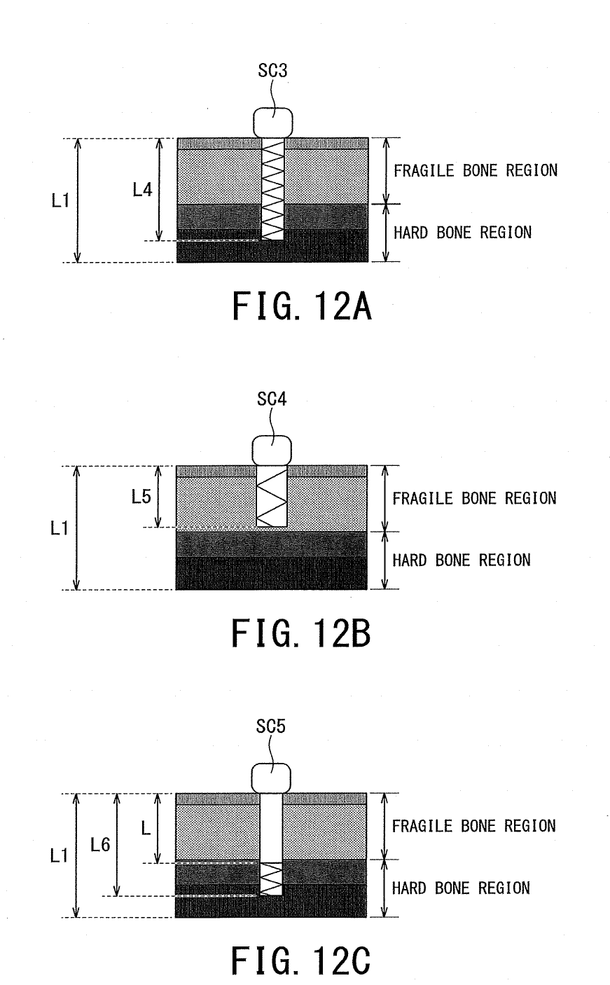

FIG. 11 is a schematic diagram illustrating the screw direction to be inserted. FIG. 11 is a sagittal cross-section of the fracture-site image, similarly to the upper part of FIG. 5. The arrows AR1 to AR5 shown in FIG. 11 indicate the screw directions. As shown by the arrows AR3 and AR4 in FIG. 11, the screw direction is not limited to the direction perpendicular to the surface of the plate.

The arrows AR1, AR2 and AR5 show the case of inserting the screw in the direction perpendicular to surface of the plate. On the other hand, the arrows AR3 and AR4 show the case of inserting the screw in the direction that is perpendicular to the fracture line in the fracture site X. In this manner, when the target bone is separated by the fracture line into the bone B1 and the bone B2, the bones B1 and B2 are fixed to each other such that the bones B1 and B2 do not shift from each other. In such a case, it is necessary to insert the screw at various angles according to the direction of the fracture line.

When a screw is inserted in a direction that is not perpendicular to a plate surface, a plate to which a multi-shaft screw can be fixed is used. In the case of a plate that can be used in combination with a multi-axis screw, the inside of its hole, into which a screw is inserted, is curved in a direction non-perpendicular to the plate surface, and this bending allows the screw to be inserted at a predetermined angle with respect to the plate surface.

Further, when the screw direction for each screw position is inputted to the medical image processing apparatus 5 prior to determination of bone fragility, it is possible to determine the bone fragility in the screw direction. That is, in the case of inserting the screw in a direction non-perpendicular to the plate surface, the fragility determining function 541 may determine fragility of a cylindrical region included in the target bone in the direction specified by a user, in a manner similar to the above-described manner. The function of designating the screw position and the screw insertion direction is the function of the processing circuitry 54.

Although a description has been given of the case where the fragility determining function 541 determines fragility of a predetermined solid, a surface of a bone, or a predetermined region of a cross-section, embodiments of the present invention are not limited to such an aspect. For instance, the fragility determining function 541 may stepwisely determine bone fragility in the depth direction of the screw direction. Additionally, the fragility determining function 541 may hierarchically determine bone fragility in respective cross-sections of the target bone in such a manner that the respective cross-sections are parallel to the screw direction at intervals of, for example, several millimeters.

The screw selecting function 543 may identify the fixed position of the screw for each type of screw on the basis of bone fragility in the depth direction of the screw direction so as to propose the fixed position to the user. In addition, the screw database 512 may contain information on the fixed position of a screw, and the screw selecting function 543 may select applicable screws according to the fixed position.

Each of FIGS. 12A to 12C is a schematic cross-sectional view of the target bone in the screw direction depicted similarly to FIGS. 9A and 9B for illustrating types of screws and fixed positions. In FIGS. 12A to 12C, bone fragility is hierarchically determined in the depth direction of the screw insertion direction, and is indicated by the color map based on the Misch classification. Each of FIGS. 12A to 12C shows the target bone composed of four bone regions that are classified into D3, D4, D2, and D1 from the top under the Misch classification. That is, FIGS. 12A to 12C indicate that the upper half of the target bone is fragile and the lower half of the target bone is hard.

FIG. 12A shows a case of the screw SC3 that exerts its fixing force at a hard position of a bone. For instance, in the target bone of FIGS. 12A to 12C, since the second top region classified as D4 is relatively larger in area that the other regions, it is assumed that fragility determining function 541 determines that the fragility of the entire target bone at the inserted position of the screw is comprehensively determined as D3. In this case, the screw SC3 that exerts its fixing force with respect to a hard bone is not selected as an applicable screw.

However, when the fragility of the target bone is determined in the depth direction as shown in FIG. 12A to FIG. 12C and it is clear that there is a hard region in the deeper region of the target bone, the screw selecting function 543 may select the screw SC3, which exerts its fixing force with respect to a hard bone, as an applicable screw under the condition that the length L4 of the shaft portion of the screw SC3 is longer than the distance from the surface of the target bone to the hard region of the target bone.

FIG. 12B shows a case of the screw SC4 that exerts its fixing force at a soft position of a bone. In this manner, by specifying bone fragility in the depth direction, it is possible to insert an applicable screw suitable to the character of the target bone into an appropriate position. In addition, the range of selecting screws is expanded.

FIG. 12C shows a case of an incomplete screw in which the thread-cutting part exists only in a part of the shaft. For instance, by specifying bone fragility in the depth direction, it is possible to determine the optimum screw in terms of the position of the thread-cutting part (for example, which part of the shaft should be formed as a thread-cutting part in the case of the optimum screw). Additionally, it is also possible to determine whether the thread-cutting part of the incomplete screw is inserted into the hard region of the target bone or not.

In this manner, the screw selecting function 543 may select the type of screw and the screw length on the basis of bone fragility in the depth direction. In addition, the screw selecting function 543 may set the priority for each screw on the basis of the fragility of the target bone, the thickness of the target bone, and the character of the screw and may rearrange the screws in the applicable screw list in descending order of the priority so as to cause the display 52 to display the rearranged list.

For instance, priority may be set on the basis of the physical features such as strength of the fixing force among features of the screw and the applicable screw list may be rearranged and displayed. Additionally, priority may be set on the basis of geometric features such as length of the screw, and the applicable screw list may be rearranged and displayed. Further, priority may be set on the basis of applicability (for example, compatibility or adaptability) between the plate and the screw, for example, whether it is manufactured by the same manufacturer or not and whether the material is the same or not.

Moreover, on the basis of screw combinations having been preset according to the selected plate type and the fixing method, the screw selecting function 543 may determine whether the screw type or the number of screws necessary for treatment is selected or not. Furthermore, the screw selecting function 543 may determine the fixing force between the plate and the bone by reflecting the fixing force of the entirety of the selected screw and then determine whether the screw capable of exerting the fixing force effective for treatment is selected or not, on the basis of the determined fixing force between the plate and the bone.

### 3. First Modified Example

The screw selecting function 543 shown in FIG. 2 may select an appropriate screw, and display, as a guide, a limit value (maximum value) of pressing force [N] in the axial direction during fastening the screw on the display 52. Accordingly, the screw selection function 543 provides it to the user. Unskilled users tend to push the screw stronger in comparison with work by experts at the time of screw fastening work (fastening work of the tapping screw). Therefore, there is a possibility that the screw and the bone may be destroyed by the screw fastening work by the unskilled users.

Therefore, the medical image processing apparatus 5 stores in advance a pressing force database (not shown) indicating a limit value of pressing force of each screw in the memory 51. The screw selecting function 543 acquires a limit value of pressing force corresponding to the selected screw from the pressing force database, and displays it on the display 52. Each limit value of pressing force in the pressing force database is, first, obtained from a time transition of pressing force during fastening work of each screw, the time transition being described in the literature and the like. Each limit value of pressing force in the pressing force database is, secondly, obtained by measuring a time transition of pressing force at the time of screw fastening work of each screw by the experts.

For instance, a first value indicating the maximum pressing force based on the time transition of pressing force in each screw is set as the limit value of pressing force in the pressing force database. Alternatively, for instance, a second value obtained by multiplying the first value by a safety coefficient φ (0<p<1) is set as the limit value of pressing force in the pressing force database. The time transition of pressing force is measured for each screw using a general pressing force measuring device.

Note that the limit value of pressing force in the pressing force database is not limited to the first or second value described above. For example, the limit value of pressing force in the pressing force database may be a third value obtained by multiplying the first value by a coefficient q (q>0), or a fourth value obtained by multiplying the second value by the coefficient q. The coefficient q is determined depending on the fastened portion, that is, the characteristic of the bone, and, for instance, determined according to at least one of bone type (for example, femur), age of the object P and bone density of the object P. With respect to the coefficient q, a pressing force database (not shown) indicating the coefficient q according to the characteristic of the bone may be stored in advance in the memory 51.

The limit value of pressing force (for example, the above-mentioned first to fourth values) is provided to the user with respect to the selected screw. Therefore, for the user, it is possible to adjust the pressing force applied to the screw during fastening the screw to an appropriate strength. As a result, it is possible to suppress destruction of the screw itself during fastening (and after fastening), and to also suppress damage to the bone.

Further, although the screw selecting function 543 has been described in the case of providing the limit value of pressing force during the fastening work on the selected one screw after the screw selection to the user, it is not limited to that case. For instance, the screw selecting function 543 may also provide the limit value of pressing force at the time of fastening work of each screw to the user. As a result, the screw selecting function 543 can select, when the screw is selected, a screw in consideration of the limit value of pressing force at the time of fastening work.

### 4. Second Modified Example

The index indicating the degree of fragility of the bone is not limited to the Misch classification based on the CT value. For instance, the index indicating the degree of fragility of the bone may be the bone density obtained from dual images acquired by dual energy imaging. The Dual energy imaging is a method of acquiring CT images of two different energy levels.

In addition, the case where the index indicating the degree of fragility of the bone is determined based on the fracture-site image or the like generated by the X-ray CT apparatus 2 which is the modality has been described, but the present invention is not limited to that case. For instance, it may be determined based on a fracture-site image or the like generated by a magnetic resonance imaging apparatus (MRI) (not shown) as the modality.

According to at least one embodiment described above, it is possible to assist a user in selecting a screw to be inserted into a target bone.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A medical image processing apparatus configured to support selection of a screw for fixing a plate to a bone, the apparatus comprising processing circuitry configured to:
acquire a medical image including a target bone to which the plate is to be fixed;
identify the target bone based on the medical image;
calculate spatial distribution indicating fragility degree of the target bone, based on pixel values of the target bone; and
select a screw to be embedded in the target bone, based on the spatial distribution.

2. The medical image processing apparatus according to claim 1, further comprising a memory configured to store a screw table in which types of screws and fragility degree of each bone are associated with each other, wherein
the processing circuitry is configured to select a screw to be embedded in the target bone, based on the screw table.

3. The medical image processing apparatus according to claim 1, wherein
the processing circuitry is configured to
designate a position where a selected screw is embedded in the target bone,
calculate length of the target bone along a direction parallel to a direction in which the selected screw is embedded at the position, and
determine whether the selected screw is to be embedded in the target bone or not.

4. The medical image processing apparatus according to claim 1, wherein
the processing circuitry is configured to determine fragility degree of the target bone based on CT values.

5. The medical image processing apparatus according to claim 1, wherein
an index indicating the fragility degree of the target bone is Misch classification; and
the processing circuitry is configured to select a screw to be embedded in the target bone, based on the Misch classification.

6. The medical image processing apparatus according to claim 2, wherein
the table includes data of strength of fixing force for each type of screw when the plate is fixed to the target bone, and
the processing circuitry is configured to select a screw to be embedded in the target bone, based on the fragility degree of the target bone and the strength of fixing force.

7. The medical image processing apparatus according to claim 6, wherein
the table includes such data of strength of fixing force that a locking screw is included as a screw having strong fixing force.

8. The medical image processing apparatus according to claim 1, wherein
the processing circuitry is configured to
determine fragility of the target bone in a predetermined range by statistically processing fragility of the target bone determined for each pixel within the predetermined range, and
select a screw to be embedded within the predetermined range.

9. The medical image processing apparatus according to claim 1, wherein
the processing circuitry is configured to
determine fragility of entirety of the target bone, to which the plate is fixed, based on fragility of the entirety of the target bone determined for each pixel, and
select a screw to be embedded in the entirety of the target bone.

10. The medical image processing apparatus according to claim 1, wherein
the processing circuitry is configured to
determine fragility degree of the target bone in a depth direction in which the target screw is embedded, and
select a screw to be embedded in the target bone, based on the fragility degree of the target bone in a depth direction.

11. The medical image processing apparatus according to claim 1, wherein
the processing circuitry is configured to weight fragility of a bone determined for each pixel, based on distance from a fracture line.

12. The medical image processing apparatus according to claim 1, wherein
the processing circuitry is configured to display, as a guide, a limit value of pressing force during fastening the screw to a display.

13. The medical image processing apparatus according to claim 12, wherein
the limit value of pressing force is a first value indicating a maximum pressing force based on a time transition of pressing force of the screw, or a second value obtained by multiplying the first value by a safety coefficient.

14. The medical image processing apparatus according to claim 12, wherein
the limit value of pressing force is a first value indicating a maximum pressing force based on a time transition of pressing force of the screw, or a value obtained by multiplying a second value obtained by multiplying the first value by a safety coefficient, by a coefficient, the coefficient being determined according to at least one of a type of the target bone, an age of the object, and the target bone density.

15. A medical image processing method for supporting selection of a screw for fixing a plate to a bone, the method comprising:
acquiring a medical image including a target bone to which the plate is to be fixed;
identifying the target bone based on the medical image;
calculating spatial distribution indicating fragility degree of the target bone, based on pixel values of the target bone; and
selecting a screw to be embedded in the target bone, based on the spatial distribution.

16. A medical image diagnostic apparatus configured to support selection of a screw for fixing a plate to a bone, the apparatus comprising:
an imaging apparatus configured to irradiate an object with an X-ray and to acquire an X-ray image including a target bone to which the plate is to be fixed; and
processing circuitry configured to
identify the target bone based on the medical image,
calculate spatial distribution indicating fragility degree of the target bone, based on pixel values of the target bone, and
select a screw to be embedded in the target bone, based on the spatial distribution.
